(19) United States
(12) Patent Application Publication
(10) Pub. No.: US 2009/0312524 A1
(43) Pub. Date: Dec. 17, 2009

(54) COMPOSITIONS AND METHODS COMPRISING COLLAGEN

(75) Inventor: Nels J. Lauritzen, Piscataway, NJ (US)

Correspondence Address: KALOW & SPRINGUT LLP, 488 MADISON AVENUE, 19TH FLOOR, NEW YORK, NY, 10022, US

(73) Assignee: ALTERNATIVE SOURCED COLLAGEN, LLC, Piscataway, NJ (US)

(21) Appl. No.: 10/580,904

(22) PCT Filed: Nov. 29, 2004
(86) PCT No.: PCT/US04/40034

§ 371 Date: Jul. 29, 2008

Related U.S. Application Data
(60) Provisional application No. 60/525,448, filed on Nov. 28, 2003.

(51) Int. Cl. C07K 14/78; C07K 1/14

(52) U.S. Cl. 530/356; 435/273

(57) ABSTRACT

In various embodiments, a collagen product is provided that is derived from an animal, the collagen product comprises precipitated collagen that is substantially pure. In various embodiments, the collagen is obtained from a marine animal and does not contain prions or viruses. In various embodiments, the collagen can be made or incorporated into collagen films, collagen membranes, cosmetic collagen masks, collagen sponges, gelatin, hemostasis sponges, lyophilized foams, collagen injections, artificial skins and dura, bones, cartilage, screws, shafts, stems, or tube guides.

This application claims the benefit of the filing date of Provisional Application No. 60/525,448, filed Nov. 28, 2003, entitled “Purified Marine Collagen and Products thereof” this entire disclosure is hereby incorporated by reference into the present disclosure.

### BACKGROUND OF THE INVENTION

Collagen is a fibrous protein that provides structural support for bones, skin, tendons, ligaments, and blood vessels and is the most abundant protein in the body. For many years, collagen has been widely used in medical, pharmaceutical and cosmetic products. The structural compatibility of collagen within the human body makes it an invaluable component in many treatment modalities including wound and tissue healing, bone regeneration, and biomedical implants. This is why research is continually exploring new applications for collagen products, expanding into such areas as drug delivery, collagen graft coatings and vascular stents, and many other applications.

The majority of collagen on the worldwide market today is obtained from hoofed or ungulate animal remains e.g., cows, goats, pigs, etc. Although hoofed animals remains provide an abundant and inexpensive source of collagen, many collagen products using these animal remains are not highly purified and have the potential to cause harmful inflammatory or immune reactions.

In addition, some conventional collagen from hoofed animals, such as type I bovine collagen, has raised fears concerning contamination of the collagen with deadly viruses or prions. Typically, prions or proteinaceous infective particles, in particular, may cause life-threatening brain diseases generally referred to as Transmissible Spongiform Encephalopathies (TSEs). Some known types of TSEs include Bovine Spongiform Encephalitis (BSE), CJD (Creutzfeldt-Jalcob disease), CVVD (Chronic Wasting Disease), and multiple other titles. These types of TSEs are fatal and to date have no cure.

The fears about viral and prion contamination of collagen have grown globally. For instance, the European Union has put companies on notice that steps need to be taken to insure that the collagen used is free from potential prion and viral contamination. In the United States, the FDA has implemented stringent regulations to insure the safety of medical devices containing bovine collagen. Manufacturers of collagen are ever vigilant to develop test methods to monitor, eliminate and in short, deal with the viral or prion contamination problem.

Some manufacturers promote recombinant and synthetic sources of collagen as alternatives to animal-derived collagen because they may reduce the risk of TSEs and other diseases. However, obtaining collagen from recombinant and synthetic sources is cumbersome, expensive, and time consuming.

New compositions and methods are needed for providing highly purified collagen, no matter what the source, that reduces the risk of harmful inflammatory or immune reactions. Collagen obtained from non-hoofed animals, which is free from deadly virus or prion contamination is needed.

### SUMMARY OF THE INVENTION

In various embodiments, compositions and methods are provided that allow the production of collagen that is highly purified and reduces or eliminates potential for harmful inflammatory or immune reactions.

In various embodiments, collagen compositions and methods are provided from a source that has no known association of transmissible prions or viral contaminants.

In one embodiment, a collagen product is provided derived from an animal, the collagen product comprising precipitated collagen from an acidic collagen dispersion, wherein the precipitated collagen is substantially pure collagen.

In another embodiment, a collagen product is provided derived from an animal without hoofs, wherein the collagen product comprises two alpha 1(I) chains and one alpha 2(I) chain heterotrimer of collagen or type I collagen and is not derived from skin of the animal.

In one exemplary embodiment, a method is provided for obtaining a collagen product from a marine animal comprising: a) isolating two alpha 1(I) chains and one alpha 2(I) chain heterotrimer of collagen or type I collagen from a marine animal, wherein the collagen is not isolated from skin; and b) recovering the two alpha 1(I) chains and one alpha 2(I) chain heterotrimer of collagen or type I collagen to obtain the collagen product.

In another exemplary embodiment, a method is provided for obtaining a collagen product from an animal, comprising: alkalinizing an acidic collagen dispersion containing collagen from the animal; and neutralizing the alkalinized collagen dispersion to precipitate the collagen; and recovering the collagen to obtain the collagen product.

In yet another exemplary embodiment, a method is provided for obtaining collagen fibers from an animal, comprising: a) adding an enzyme to collagen particles obtained from the animal so as to substantially remove non-collagenous materials from the collagen particles, b) inactivating and washing the enzyme from the collagen particles; c) alkalinizing the collagen particles and neutralizing the alkalinized collagen particles with an acid to obtain a collagen dispersion, d) precipitating collagen fibers from the collagen dispersion to obtain the collagen fibers.

Additional features and advantages of various embodiments will be set forth in part in the description that follows, and in part will be apparent from the description, or may be learned by practice of various embodiments. Other advantages of various embodiments will be realized and attained by means of the elements and combinations particularly pointed out in the description and appended claims.

### BRIEF DESCRIPTION OF THE FIGURES

FIG. 1 illustrates purified collagen fibers from tuna tendons obtained by the novel precipitation method. The fibers are uniquely shard like and transparent. The fibers have firm density and strong tensile strength.

FIG. 2 illustrates a schematic of purified collagen fibers from tuna tendons obtained by the novel precipitation method draped over a spatula. The fibers are uniquely shard like and transparent due to swelling caused by the retention of water.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION OF THE INVENTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a monomer” includes two or more monomers.

Reference will now be made in detail to certain embodiments of the invention. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

Collagen

In various embodiments, the collagen is obtained from a native animal source. Some native collagen animal sources include, but are not limited to, avians or birds, ungulates or hoofed mammals, such as for example, horses, cows, goats, pigs, sheep, deer, non-ungulates or mammals without hoofs; marsupials, such as for example, opossums, kangaroos, wombats, and bandicoots; marine animals or any animal that lives in water, such as for example, whales, dolphins, swordfish, tuna, shark, mahimahi, sailfish, marlin, yellowtail, escolar, lancet fish, mackerel, salmon, cod, flounder, bass, or sturgeon; amphibians, such as for example, frogs, toads, salamanders; reptiles, such as for example, snakes, crocodiles, alligators, or combinations thereof.

In preferred embodiments, the collagen is obtained from marine animals because no known association with TSE prions or viral contaminants have been demonstrated.

In various embodiments, the collagen can be obtained from any body part of the animal that has collagen. For example, collagen may be obtained from skin, tendons, ligaments, or bone, or the like. In a preferred embodiment, the collagen source is from a marine animal and the body part is from the tendon, such as for example, caudal, caudal ray, pectoral, and/or inter-costal tendon.

Presently, about twenty distinct collagen types have been identified in vertebrates, including bovine, ovine, porcine, chicken, marine, and human sources. Generally, the collagen types are numbered by Roman numerals, and the chains found in each collagen type are identified by Arabic numerals. Detailed descriptions of structure and biological functions of the various different types of naturally occurring collagens are generally available in the art.

Type I collagen is the major fibrillar collagen of bone and skin, comprising approximately 80-90% of an animal's total collagen. Type I collagen is the major structural macromolecule present in the extracellular matrix of multicellular organisms and comprises approximately 20% of total protein mass. Type I collagen is a heterotrimeric molecule comprising two alpha 1(I) chains and one alpha 2(I) chain. Other collagen types are less abundant than type I collagen, and exhibit different distribution patterns. For example, type II collagen is the predominant collagen in cartilage and vitreous humor, while type III collagen is found at high levels in blood vessels and to a lesser extent in skin.

Type II collagen is a homotrimeric collagen comprising three identical alpha 1(II) chains. Type III collagen is a major fibrillar collagen found in skin and vascular tissues. Type III collagen is a homotrimeric collagen comprising three identical alpha 1(III) chains.

Type IV collagen is found in basement membranes in the form of sheets rather than fibrils. Most commonly, type IV collagen contains two alpha 1 (IV) chains and one alpha 2(IV) chain. Type V collagen is a fibrillar collagen found in, primarily, bones, tendon, cornea, skin, and blood vessels. Type V collagen exists in both homotrimeric and heterotrimeric forms. One form of type V collagen is a heterotrimer of two alpha 1 (V) chains and one alpha 2(V) chain. Another form of type V collagen is a heterotrimer of alpha 1(V), alpha 2(V), and alpha 3(V) chains. A further form of type V collagen is a homotrimer of alpha 1(V).

Type VI collagen has a small triple helical region and two large non-collagenous remainder portions. Type VI collagen is a heterotrimer comprising alpha 1 (VI), alpha 2(VI), and alpha 3(VI) chains and is found in many connective tissues. Type VII collagen is a fibrillar collagen found in particular epithelial tissues, and is a homotrimeric molecule of three alpha 1(VII) chains.

Type VIII collagen can be found in Descemet's membrane in the cornea and is a heterotrimer comprising two alpha 1(VII) chains and one alpha 2(VIII) chain. Type IX collagen is a fibril-associated collagen found in cartilage and vitreous humor, and is a heterotrimeric molecule comprising alpha 1(IX), alpha 2(IX), and alpha 3 (IX) chains. Type IX collagen has been classified as a FACIT (Fibril Associated Collagens with Interrupted Triple Helices) collagen, possessing several triple helical domains separated by non-triple helical domains.

Type X collagen is a homotrimeric compound of alpha 1 (X) chains. Type X collagen has been isolated from, for example, hypertrophic cartilage found in growth plates. Type XI collagen can be found in cartilaginous tissues associated with type II and type IX collagens, and in other locations in the body. Type XI collagen is a heterotrimeric molecule comprising alpha 1(XI), alpha 2(XI), and alpha 3(XI) chains.

Type XII collagen is a FACIT collagen found primarily in association with type I collagen. Type XII collagen is a homotrimeric molecule comprising three alpha 1(XII) chains. Type XIII collagen is a non-fibrillar collagen found, for example, in skin, intestine, bone, cartilage, and striated muscle.

Type XIV is a FACIT collagen characterized as a homotrimeric molecule comprising alpha 1(XIV) chains. Type XV collagen is homologous in structure to type XVIII collagen. Type XVI collagen is a fibril-associated collagen, found, for example, in skin, lung fibroblast, and keratinocytes. Type XVII collagen is a hemidesmosal transmembrane collagen, also known at the bullous pemphigoid antigen. Type XVIII collagen is similar in structure to type XV collagen and can be isolated from the liver.

Type XIX collagen is believed to be another member of the FACIT collagen family, and has been found in mRNA isolated from rhabdomyosarcoma cells. Type XX collagen is a newly found member of the FACIT collagenous family, and has been identified in chick cornea.

The term “collagen” as used herein refers to any one of the known collagen types, including collagen types I through XX, as well as to any other collagens. The term “collagen” specifically encompasses variants and fragments thereof, and functional equivalents and derivatives thereof, which preferably retain at least one structural or functional characteristic of collagen. So, for example, the term “bovine type I collagen” refers to a homotrimeric or heterotrimeric collagen comprising bovine type I collagen chains, and to any corresponding fragment, variant, fiber, functional equivalent, or derivative thereof.

In various embodiments, the precipitated collagen can comprise type I bovine collagen. In various embodiments, the precipitated collagen comprises two alpha 1(I) chains and one alpha 2(I) chain heterotrimer of collagen and is not derived from skin of an animal. In various embodiments, the collagen is derived from the skin of an animal and may comprise the two alpha 1 (I) chains and one alpha 2(I) chain and alpha-3 heterotrimer.

In one preferred embodiment, the collagen is obtained from the tendons of a tuna, which is a rich source of the two alpha 1(I) chains and one alpha 2(I) chain heterotrimer that is typical of vertebrate bovine type I collagen. The amino acid analysis of the collagen obtained from the tendons of the tuna is shown in Table I and is similar to type I bovine collagen. The mole percent of the collagen comprises about: hydroxyproline 7.192, aspartic acid 3.634, threonine 3.303, serine 2.507, glutamic acid 9.353, proline 17.134, glycine 30, alanine 19.598, valine 2.922, methionine 1.711, isoleucine 1.034, leucine 2.356, tyrosine 0.315, phenylalanine 1.291, hydroxylysine 0.437, histidine 0.906, and lysine 4.511.

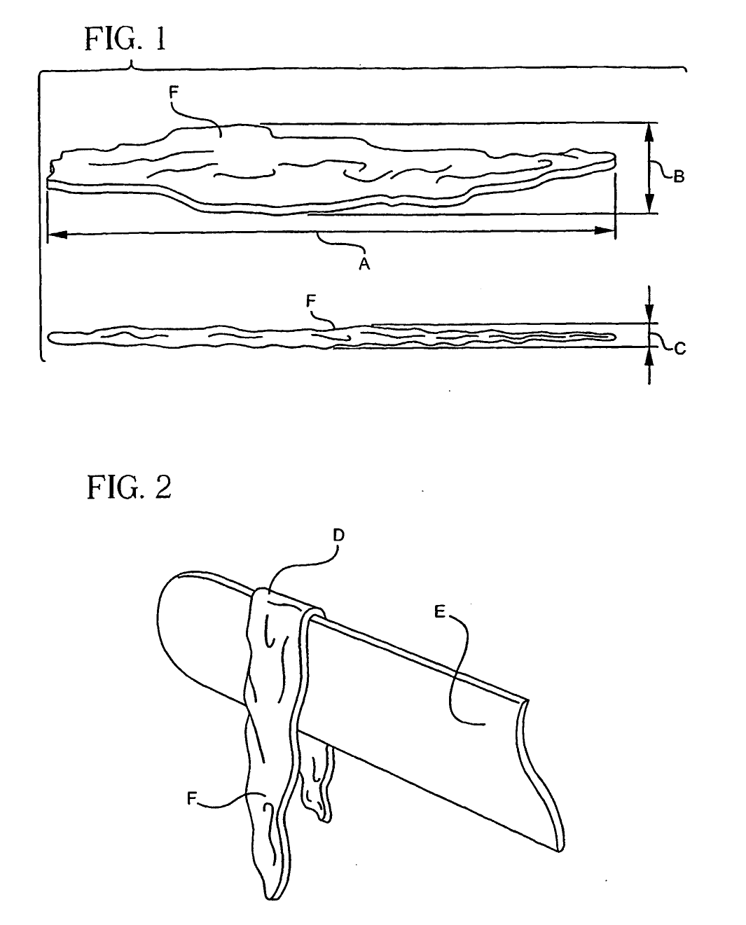

In various embodiments, the collagen can be a fiber or a filamentary material that can be entangled together and provides firm density and tensile strength. FIGS. 1 and 2 show preferred collagen fibers that appear, for example, as uniquely shard-like and transparent structures.

In various embodiments, a collagen product is provided that comprises precipitated collagen. Precipitated collagen includes collagen that is separated from an acidic dispersion of collagen. Typical acid dispersions of collagen have a pH of less than 7.0 and have collagen particles dispersed in liquid. Collagen particle include less than or the entire collagen protein. Collagen particle can encompass any one of the known collagen types, including collagen types I through XX, as well as to any other collagen fragment, fiber, variant, functional equivalent, or derivative thereof.

Preferably, the collagen after it is isolated is substantially pure, which means that the collagen is free not only of other proteins, but also of other materials used in the isolation and identification of the collagen, such as, for example, enzymes, reagents, non-collagenous materials, telopeptides, prions, viruses, glycoproteins, lipids, and/or telopeptides that may cause disease, inflammatory and/or immunological reactions. In various embodiments, the collagen is at least 90% free, preferably at least 95% free and, more preferably, at least 99% free of such materials. Collagen is considered to be substantially pure if it is at least 90%, preferably at least 95%, and more preferably at least 99% pure.

Collagen product includes any product containing at least one collagen, including collagen types I through XX, as well as to any other collagens fragment, fiber, variant, functional equivalent, or derivative thereof, and any derived product, including hydrolyzates. Typically, collagen products contain from 0.01% to 100% by weight of collagen. Optionally, the collagen may be deodorized by methods known in the art.

In various embodiments, the collagen used in the collagen product may be crosslinked by thermal dehydration, chemical, and/or light treatment. If, the thermal dehydration is used, it is carried under vacuum at a temperature between 60° C. to about 130° C.

In various embodiments, the collagen can be incorporated into a matrix to form the collagen product and used to prevent or treat diseases or conditions. Suitable collagen matrices that the collagen can be incorporated into include, but are not limited to, collagen film, collagen membranes, cosmetic collagen masks, collagen sponges, microfibrillar collagen, hemostasis sponges, lyophilized foams, collagen injections, artificial dura or artificial skin.

In various embodiments, the collagen product of the present invention comprises a matrix provided in the form of a collagen sponge, or a non-woven matrix, felt or film. In addition, the collagen product can be provided in the form of a composite of any two or more of the foregoing forms, such as, e.g., a film/sponge or a film/sponge/film.

Typically, a collagen sponge can be prepared by lyophilization of a collagen dispersion having a concentration of between 0.1 and 10% solids (w/w) and more preferably at least 0.75% solids. A volume of the dispersion is poured into a suitable (preferably non-stick) tray to provide a sponge having a suitable shape. Preferably, the sponge has a thickness from about 2.5 mm to about 5 mm, and more preferably 3 mm. The dispersion is then frozen and lyophilized for about 1 to about 48 hours. It is known in the art that the density of the dispersion and the lyophilization cycle dictate the sponge density and pore size. For example, the sponge density may be preferably about 0.0001 mg/mm³ to about 0.12 mg/mm³, more preferably about 0.009 mg/mm³.

In various embodiments, collagen sponges preferably have pores of a sufficient size and quantity to permit wound healing and/or growing of tissue. In various embodiments, the pore size preferably ranges from about 10 μm to about 500 μm, more preferably from about 50 μm to about 150 μm, with surface pores being smaller than cross-sectional (internal) pores. In particularly preferred embodiments, the surface pores range in diameter from about 30 μm to about 150 μm, with about 70 μm being most preferred, and the cross-sectional pores range in diameter from about 50 μm to about 300 μm with about 150 μm being most preferred.

A collagen film can be provided by casting a dispersion of collagen having a collagen concentration of about 0.1 to about 10% solids (w/v) and, optionally, about 0.005 to 0.5% (w/w on collagen solids) of a suitable biocompatible plasticizer, such as glycerine. Preferably, the plasticizer concentration is about 0.1% and the collagen concentration is about 1%, more preferably 0.75%. A volume of the dispersion is poured into a suitable non-stick container and evaporated to provide a film having a thickness of about 0.05 to about 2.0 mm, preferably about 0.5 mm. The film can be cross-linked with heat or a suitable chemical cross-linking agent or light. As with the sponge, collagen felt, films, and non-woven embodiments of the present invention, preferably have pores of a sufficient size and quantity to permit growing of tissue and infiltration of cellular elements therein. As used herein, non-woven matrix includes a random distribution of collagen fibers derived from collagen dispersions.

In various embodiments, the product can also be provided in the form of a combination of any two or more of the foregoing forms. In such an embodiment, all of the forms need not be sufficiently porous to promote tissue growth there through, as long as at least one sufficiently porous form is accessible to the growing tissue.

In various embodiments, it is particularly preferred to provide the collagen product in the form of a laminate of a collagen sponge, collagen textile or woven/knitted cloth or a collagen film. This laminate, which can be formed, e.g., by laminating a collagen sponge to a collagen film with a biocompatible adhesive or polymer (including collagen), by forming a sponge on a film, or by forming a film on a sponge, possesses the elevated water impermeability and suturability of a film, and the elevated porosity of a sponge, which facilitates tissue growth therethrough. Similarly, a sandwich-type laminate can be provided by disposing a collagen sponge between opposing sheets of collagen film.

In various embodiments, the film can have a shape that perfectly mirrors the underlying surface of the sponge to which it is bonded. In other embodiments, the bonding surface of the sponge does not identically correspond in shape and/or size to the bonding surface of the film. For example, a film can be sandwiched between two opposing sponges which do not overhang the ends of the film (thus leaving the edges of the film uncovered), or a film can be sandwiched between two opposing sponges which overhang the ends of the film and are bonded together as well as to the intermediate film (thus completely encasing the film in the sponges).

In various embodiments, sponge/film laminates are prepared by casting a collagen film; drying the film; casting a collagen slurry onto the dried film; lyophilizing the slurry/film combination; and cross-linking the lyophilized laminate product by exposing it to vapors from an aqueous formaldehyde solution (preferably having a 9.6% formaldehyde concentration) for about ninety minutes at about 25° C., followed by forced air ventilation for about one hour. The collagen film and slurry are preferably cast from lactic acid derived collagen fibers. Such fibers are produced by a process comprising dispersing a virus and prion free collagen source (e.g., alkali-treated marine tendon slices) in an aqueous solution of lactic acid (preferably about 85%), homogenizing the dispersion, filtering the homogenized lactic acid dispersion, and precipitating collagen fibers from the homogenized lactic acid dispersion by addition of aqueous ammonium hydroxide (preferably 0.35%) sufficient to adjust the pH to about 4.6-4.9.

Lactic acid derived/ammonium hydroxide precipitated collagen fibers are much longer than fibers produced by mechanical/chemical disruption of raw bovine tendon material. During ammonium hydroxide precipitation, the collagen fibers re-coil and are therefore longer. Longer fibers provide greater strength to the final product. The enhanced strength of collagen products produced according to this particularly preferred method can be sufficiently strong to be watertight without the need for cross-linking, thus allowing the degree of cross-linking to be selected based on the desired rate of bioresorption.

The product can include biocompatible and/or bioresorbable materials other than collagen, although collagen is most preferred. For example, in certain embodiments it is advantageous to laminate the collagen matrix to a non-collagen film, such as a 50:50 dl lactide:co-glycolide polymer having a molecular weight of about 75,000, more preferably about 100,000. Additional suitable polymers include, e.g., biocompatible and/or bioresorbable lactides, glycolides, and copolymers thereof, polycaprolactones, polyethylene carbonate, tyrosine polycarbonates, tyrosine polyacids, and polyanhydrides. The molecular weight of the polymer is preferably about 5000 to about 500,000.

In various embodiments of the present invention, gelatin may be obtained from the isolated and/or substantially pure collagen. Gelatin is a derivative of collagen, a principal structural and connective protein in animals. Gelatin can be derived from denaturation of collagen and contains polypeptide sequences having Gly-X-Y repeats, where X and Y are most often proline and hydroxyproline residues. These sequences contribute to triple helical structure and affect the gelling ability of gelatin polypeptides. Gelatin can be obtained from the animal collagen source. The biophysical properties of gelatin make it a versatile material, widely used in a variety of applications and industries. Gelatin is used, for example, in numerous pharmaceutical and medical, photographic, industrial, cosmetic, and food and beverage products and processes of manufacture. Gelatin is thus a commercially valuable and versatile product.

“Gelatin” as used herein refers to any molecule having at least one structural and/or functional characteristic of gelatin. Gelatin is currently obtained from collagen derived from the animal (e.g., bovine, porcine, chicken, equine, marine) sources, e.g., bones, skin, and tendons. The term gelatin encompasses both the composition of more than one polypeptide included in a gelatin product, as well as an individual polypeptide contributing to the gelatin material.

Methods, processes, and techniques of producing gelatin from collagen include denaturing the triple helical structure of the collagen utilizing detergents, heat or denaturing agents. Additionally, these methods, processes, and techniques include, but are not limited to, treatments with strong alkali or strong acids, heat extraction in aqueous solution, ion exchange chromatography, cross-flow filtration and heat drying, and other methods that may be applied to collagen to produce the gelatin.

In various embodiments, the collagen is incorporated into bone, cartilage, skin, screws, shafts, stents, tube guides or combinations thereof for prevention or treatment of a disease or condition.

Making Collagen

In various embodiments, the collagen may be prepared by obtaining a native animal source of collagen. Some native collagen animal sources include, but are not limited to, avians or birds, ungulates or hoofed mammals, such as for example, horses, cows, goats, pigs; sheep, deer; non-ungulates or mammals without hoofs; marsupials, such as for example, opossums, kangaroos, wombats, and bandicoots; marine animals include any animal that lives in water, such as for example, whales, dolphins, swordfish, tuna, shark, mahimahi, sailfish, marlin, yellowtail, escolar, lancet fish, mackerel, salmon, carp, cod, flounder, bass, or sturgeon; amphibians, such as for example, frogs, toads, salamanders; reptiles, such as for example, snakes, crocodiles, alligators, or combinations thereof. In preferred embodiments, the collagen is obtained from marine animals, because no known association with TSE prions or viral contaminants have been demonstrated.

The collagen can be obtained from any body part of the animal that has collagen. For example collagen may be obtained from skin, tendons, ligaments, or bone, fins, tails, or the like. In a preferred embodiment, the collagen source is from a marine animal and the body part is from the tendon, such as for example, caudal, caudal ray, pectoral, and/or inter-costal tendon.

In making the collagen, the body part is first mechanically or hand cleaned of fat, blood, vascular material and other extraneous matter and washed. Optionally, the body part is frozen. The cleaned and washed collagen containing material is then comminuted, generally by slicing, grinding or milling so that it easily to obtain the desired collagen.

The comminuted material is then subjected to an enzyme treatment with a proteolytic enzyme, such as ficin, pepsin, amylase, lipase, pancreatin or the like, so as to remove non-collagenous impurities and telopeptides which may cause unwanted inflammatory and/or immunological activity and/or swell the collagen by removing elastin. The amount of enzyme added to the collagen material and the conditions under which enzyme digestion takes place is dependent upon the particular enzyme being used and the part and type of the animal being used as the collagen source. For example, when using pancreatin on animal tendon, the collagen material is digested for about 1 to 2 hours at a temperature of about 20 to about 40° C. Buffers may be added to provide the optimum working environment, e.g. pH, temperature and agitation, for the selected proteolytic enzyme.

In various embodiments, after the requisite amount of time, the enzyme is inactivated by appropriate means well known in the art such as by the addition of a solution of an oxidizing agent, such as sodium chlorite, hydrogen peroxide, or the like.

In various embodiments, the enzyme deactivated collagen material is separated from the reagents by mesh or centrifugation and washed to remove enzyme, collagenous impurities, telopeptides and reagent from the collagen material or collagen fibers. Preferably, the washing is carried out with ultrafiltered and/or deionized water and optionally further washed with dilute aqueous hydrogen peroxide. In various embodiments, the pH of the wash is between about 6 to about 8.

The washed collagen material including collagen fibers, in various embodiments, is subjected to alkali treatment to remove fats/oils, contaminating glycoproteins, lipids and other non-collagenous materials by applying the appropriate pH, temperature and agitation. In various embodiments, the alkali treatment occurs at a pH of about 10 to 14, at a temperature of about 20 to about 40° C. for a period of about 15 to 48 hours, preferably about 40 hours. The alkali treatment can be carried out at, for example, in an aqueous solution of sodium hydroxide, sodium carbonate, ammonia, sodium sulfate, or the like.

In various embodiments, the alkali treated collagen material including fibers is further washed to remove fats/oils, contaminating glycoproteins, lipids and other non-collagenous materials. The washed collagen material is then treated with a suitable acid, that preferably does not cause any cross-linking of the collagen, to form a collagen dispersion. Typically, the acid will neutralize the dispersion. Suitable acids include, but are not limited to, aqueous sulfuric acid, hydrochloric acid, lactic acid, acetic acid or the like. By collagen dispersion is meant that collagen particles are dispersed in a liquid. In various embodiments, the pH of the acid dispersion can be kept at the upper limit or at the isoelectric point of the dispersion, in the range of between about 2 to about 5, more preferably between about 3 to about 5, and most preferably at about 4.6 for an alkali treated collagen.

In various embodiments, the acid treatment step swells the collagen and loosens the helical structure of collagen. In the swollen state, the collagen can be optionally blended and/or homogenized to a point where a liquid like condition appears so as to dissociate collagen fibers. The collagen dispersion can be filtered or centrifuged to further remove non-collagenous material and unswollen material. In the case of centrifugation, the sediment is discarded and the collagen dispersion supernatant is retained for further treatment.

In various embodiments, the dispersion is very pure and collagen fibers can slowly be precipitated by drop-by-drop addition of an alkali, such as for example, sodium hydroxide, sodium carbonate, ammonia, sodium sulfate, or the like. Typical pH for this precipitation step is about 7. The collagen fibers can be filtered or collected by hand.

In various embodiments for the collagen precipitation, the pH of the acidic dispersion of collagen is pH of about 3.5 as a starting point for the precipitation reaction, at about pH of 4.6 transparent shard-like structures form, these structures precipitate out of the acid dispersion at a pH of about 6.0 to about 7.0, where they are transparent fully formed, firm and stable structures. In various embodiments, the temperature for precipitation is about 20 to about 30° C.

In various embodiments, the collagen formed is transparent shard-like structures resembling flexible icicles. The collagen fibers appear like shard-like gelatin because it is thought that water is trapped within the fiber structure. The collagen fibers can be de-watered making the fiber structure more textile like and allows further removal of contaminants, such as non-collagenous material, trapped within the water.

In various embodiments, dewatering of the collagen fibers can be accomplished by, for example, centrifugation, washing with suitable drying agents, air, and/or oven drying. Suitable drying agents include, for example, non-polar solvents such as for example, acetone, alcohol, or the like. Low temperature drying, such as by air and/or oven at temperatures, for example, of about 35 to about 40° C. can remove any remaining water as the solvents flash off, and can leave substantially pure collagen in dry firm fiber form.

In general, prior art methods of obtaining collagen involve after the enzyme treatment and deactivation steps, alkali treatment at a required pH range of 13-14 to obtain the collagen fiber, which is then dispersed in acid. The prior art then homogenizes and filters the collagen dispersion to obtain swollen collagen fibers, which are then freeze-dried and incorporated into a collagen sponge.

In contrast to the prior art, in various embodiments of the present invention, the alkali treatment does not need to be as high as 13-14, for example, the alkali treatment can be accomplished at pH of about 11. Further, in various embodiments of the present invention, the acidic dispersion of collagen is subjected to separation, such as for example by centrifugation, to remove non-collagenous material and the collagen dispersion is then subjected to treatment with a second base to precipitate out the collagen (the first alkali treatment occurred before the formation of the acid dispersion). The precipitation step allows substantially pure collagen to be obtained. Prior art methods do not subject the collagen to two separate treatments with a base to precipitate the collagen and do not centrifuge the collagen dispersion, but rather freeze-dry it.

Typically, prior art methods of isolating collagen would not identify the transparent collagen material after the collagen was in an acid dispersion. Rather, prior art methods would look for whitish fibers in the dispersion and then subject the fibers to lyophilization and incorporate them into, for example, a collagen sponge or hemostat, while the collagen material that was not fiber would be discarded. Applicants have found that by precipitating the collagen material that was discarded by prior art methods, highly pure collagen can be obtained. In contrast to the prior art, Applicants have found that collagen fibers and, thus, substantially pure collagen can be obtained by the unique precipitation process.

Having now generally described the invention, the same may be more readily understood through the following reference to the following examples, which are provided by way of illustration and are not intended to limit the present invention unless specified.

### EXAMPLES

### Example 1

1.0 Caudal Tendon Preparation—Slice approximately 1000 grams of frozen tuna caudal tendon using a ‘deli’ (i.e. NBI Natsune deli slicer). Slice target thickness is 0.012 to 0.15 inches thickness. Weigh the resulting sliced tendon.

2.0 Solids on Sliced Caudal Tendon—Weigh out 2.0±0.5 gm. (wet weight) sliced tendon into weighing tins and determine solids by drying for 4 hours at 105° C. Three replicate samples are used to insure accuracy. Initial dry weight of ground caudal tendon should be @ 300 grams (assume @30% SOLIDS). This impacts chemistry mass balance for the remainder of the process.

3.0 Buffer Preparation—Prepare 10 liters of 1% NaHCO₃ solution by adding 100 grams of —NaHCO₃ to 10 liters of distilled or de-mineralized water. Then add 1N NaOH to the solution to get the pH to 8.5. (1N NaOH is prepared by dissolving 4 grams NaOH in 100 ml distilled H₂O). *Note: sequest @300 mL. of the prepared buffer to be used as an enzyme premix in the Enzyme Treatment in 4.0.

4.0 Enzymatic Treatment—Add the weighed out sliced caudal tendon to the above solution. Stabilize @ 20° C. and add 24 grams of Pancreatin 8× (Sigma) dissolved in 300 mL. of solution taken from previously prepared 10 liter batch.

5.0 Enzyme Deactivation (Ammonium Nitrate Solution)—Prepare solution of 10 liters distilled water, 1000 grams NH₄NO₃ and 12 grams NaClO₂. Observe and record pH of solution. *Note—Add NaClO₂ to solution during last 5 minutes of preparation/stirring, ideally immediately prior to adding the deactivation solution to the enzyme treatment solution. Add the ammonium nitrate deactivation solution directly to the enzyme treatment to deactivate the enzymatic activity. Stir intermittently for 1 hour at room temperature (22-25° C.).

Fiber Removal/Transfer—Separate the caudal tendon fibers from the treatment solutions by straining through a fine mesh screen (@ 1/32″ open), perforated metal strainer (i.e. China Hat) or Centrifuge the deactivated enzyme treatment solution at 5,000 rpm @15° C. for five (5) minutes.

6.0 Washing—Wash three (3) times, 15 minutes for each wash, with 5 liters of distilled water per wash using the centrifuge (5,000 rpm @15° C. for 5 minutes) to separate the fibers from the wash water after each washing. Observe and record pH reading of the washes.

*pH readings should be in the range of 6.0 to 8.0.

7.0 Alkali Treatment (Na₂CO₃ Solution)—Prepare a solution of 10 liters distilled water, 100 grams Na₂CO₃, at pH 11 and 20-25° C. Place the fibers into the 5 liters of 1% Na₂CO₃ solution at 20-25° C. for 18 hours. Agitate slowly (@70 rpm) using mechanical stirring.

8.0 Washing—Wash three (3) times, 5 minutes for each wash, with 3 liters of distilled water (adjusted to pH 8.5 using dilute NaOH) per wash using the centrifuge (5,000 rpm @15° C. for 5 minutes) to separate the fibers from the wash water after each washing. *Each wash is adjusted to pH 8.35-8.5 at the end of each wash cycle. Do not allow the pH to fall below 8.35 otherwise fibers will swell with water and later processing/drying will become very difficult and potentially of lower quality.

9.0 Solids—Squeeze out excess water, weigh the wet fiber and run a % solids on the fiber. Three samples are used to determine solids in order to provide accuracy.

10.0 Lactic Acid Treatment—Make a 0.7% dispersion using a 0.2% lactic acid (i.e. 2 ml. lactic acid per 1000 mL distilled water). Dispersion batches are made up in 3 liter batches due to current equipment limitations. Prepare the 0.7% dispersion by adding 21 grams (dry weight) of fiber to 3000 ml. distilled water containing 6 ml. lactic acid. To determine how much wet weight fiber to use in dispersion making:

Wet Weight=dry weight/% weight solids=21 grams/% solids=______ gm. wet weight caudal tendon fibers.

Keep the 0.7% dispersion cold (in refrigeration @ 8-14° C.) for one (1) hour, and allow the fibers to swell. Waring Blend (3.5 liter Waring Blender) the dispersions three times at ‘low’, speed for 7 seconds per setting for each batch while keeping the batches at 10-14° C.

11.0 Centrifugation—Using the Sorvall refrigerated centrifuge, centrifuge the dispersion for 5 minutes at 4000 rpm., at 15° C. Pour off all the supernatant into a clean 25.5 liter vessel (or one of suitable capacity) and the residue into a jar. Do this for each batch, collecting all of the supernatant in the 25.5 quart vessel and all the residue in a jar. Once all of the supernatant has been collected (possibly requiring two 25.5 liter vessels) re-precipitate the translucent fibers by adding 1N NaOH solution to the centrifuged solution to a point where pH 7.0 is reached. To 24 quarts of supernatant approximately 400 ml. 1N NaOH is needed. PH uniformity is very important. The pH of the initial supernatant is @pH 3. The fibers re-precipitate nicely at pH 7. If the solution is at a lower or higher pH than pH 7, the re-precipitated fibers are not easy to work with (i.e. are sticky and/or swollen). Centrifuge separate the re-precipitated fibers by centrifuge -5,000 rpm, @ 15° C. for 5 minutes. Collect all fibers (sediment) for further processing (washing and drying).

12.0 Washing—Wash the purified fibers three times with 4 liters distilled water to which sufficient dilute NaOH is added to raise the pH to 8.0. Each washing is adjusted to pH 8.0-8.35. *Note—Centrifuge separate fibers from cleaning water after each wash.

13.0 Isopropanol (IPA) Wash—Place the washed fibers into @ 4 liters (or sufficient IPA to completely cover/soak all fibers) of 100% isopropanol. Maintain the IPA/fiber mass at 30° C. Let the fibers remain in the isopropanol for at least two hours with intermittent gentle stirring. Centrifuge separates the fibers from the IPA wash (5,000 rpm, @ 15° C. for 5 minutes). Repeat the process for a second Isopropanol Wash.

14.0 Acetone Wash—Place the washed fibers into sufficient Acetone to cover/soak the fibers completely. Maintain the Acetone/fiber mass at 30° C. for one hour with intermittent gentle stirring. Hand squeeze the fibers to express the Acetone and repeat a second Acetone Wash. Hand pluck the fibers and dry at 30° C. overnight (≧8 hours) or until dry in a through air oven.

15.0 Shrink (melt) Temperature: Ts—Using the melt-temp apparatus, place a fiber in a glass capillary tube, add some water to the capillary tube to keep the fiber wet. Prepare three samples in the aforementioned method in order to provide adequate accuracy. Set the variable control knob on setting #4. Closely watch the fiber as the temperature starts to rise. The melt temperature is considered to be the point at which the fiber collapses. Do this three times and then take the average.

16.0 Trichloroacetic Acid Insolubles (% TCA Insolubles)—To 100 ml. distilled water add 2.5 grams tricloroacetic acid (TCA). Filter this through the glass filter paper using a Buchner funnel and collect the filtered solution. To the filtered 100 ml. 2.5% TCA add 2 grams of dry purified tendon fiber and a magnetic stir-bar in to a 250 ml. beaker. Place this beaker on a hot plate and bring the temperature up to 90° C. Keep the continuously stirred solution at 90° C. for 30 minutes. After 30 minutes at 90° C., let the solution cool. At the same time, preheat a glass filter paper at 37° C. for 30 minutes. Weigh the filter paper. Once the solution has cooled down, filter it through the preheated, pre-weighed filter paper using a Buchner funnel. Wash the filtrate with 500 ml. of distilled water. Allow the filter paper to dry in the oven (place the filter paper on an aluminum weigh dish) at 40° C. for 3 hours. Once the filter paper is dry, reweigh the filter paper. * Three samples should be processed in the aforementioned procedure in order to provide appropriate accuracy.

17.0 Comments:

### Typical Results:

Yield @ 60%

TCA Insolubles (measure of purity) @ 0.05%

Melt Temperature @ 40-50° C.

Product Application Materials manufactured by the aforementioned process are intended to be used for: Regenerative Matrices
Dura
Skin
Cartilage
Bone

Hemostasis
Microfibrillar

Lyophilized Foam
Bio-Engineered Material
   Coatings for implanted screws, shafts and stents
   Vascular and Neural tube guides

### Example 2

The amino acid analysis in mole % of the collagen isolated from the tuna caudal tendon is listed in Table 1 and is compared to Type I bovine collagen.

TABLE 1

| | Marine | Bovine | |
|---|---|---|---|
| | Mole % | Mole % | DIF |
| OH Proline | 7.192 | 9.845 | 2.653 |
| Aspartic | 3.634 | 2.791 | −0.843 |
| Threonine | 3.303 | 1.452 | −1.851 |
| Serine | 2.507 | 1.708 | −0.799 |
| Glutamic | 9.353 | 9.544 | 0.191 |
| Proline | 17.134 | 15.498 | −1.636 |
| Glycine | 20.318 | 23.057 | 2.739 |
| Alanine | 19.598 | 18.074 | −1.524 |
| Cysteine | 0 | 0 | 0 |
| Valine | 2.922 | 3.003 | 0.081 |
| Methionine | 1.711 | 1.073 | −0.638 |
| Isoleucine | 1.034 | 1.668 | 0.634 |
| Leucine | 2.356 | 2.703 | 0.347 |
| Tyrosine | 0.315 | 0.358 | 0.043 |
| Phenylalanine | 1.291 | 1.335 | 0.044 |
| OH Lysine | 0.437 | 0.739 | 0.302 |
| Histidine | 0.906 | 0.348 | −0.558 |
| Lysine | 2.58 | 2.171 | −0.409 |
| Arginine | 4.511 | 4.723 | 0.212 |
| | 101.102 | 100.09 | |

As it can be seen from Table 1, the amino acid contents of tuna caudal tendon is similar to bovine type I collagen, which means that the marine collagen will perform similar to bovine type I collagen.

### Example 3

FIGS. 1 and 2 describe fibers purified from materials secured from marine sources (e.g., tuna tendons) and using the precipitation purification process of the present invention. The preferred precipitation purification process, with minor and appropriate pH and enzyme variations, will successfully purify collagen from many sources.

In the case of land mammals, even toed ungulates can be avoided as ‘sources’ for collagen due to their association with “mad cow disease” and viral contaminants. However, non-ungulate (non hoof and single toed ungulates) mammals remain potential sources for collagen to be purified using the subject precipitation purification process. Additionally, reptilians (i.e. crocodilians), marsupials, amphibians, avians and sea mammals are known to be excellent sources of collagen for our subject process. In short, everything but hoofed mammals are good collagen source candidates.

FIG. 1 illustrates collagen fibers appear as uniquely shard like and substantially transparent structures having substance, firm density and resisting tensile deformation in the longitudinal and cross-sectional directions. The following letters represent:

‘A’—Length of the fibers formed from tuna tendon purification to date.

Range=@ ½″ to 2″

Majority between ¾ to 1.5″

### ‘B’—Width

Range=@ 1/16″ to ¾″
Majority between ⅛″ to ⅜″
   ‘C’—Thickness (Z-directional dimension)
Range=@ 1/64″ to ⅛″
Majority between 1/32″ to 1/16″

FIG. 2 illustrates the collagen fiber that appears very non conventional and as a shard-like structure due to excessive swelling caused by the retention of large amounts of water. The following letters represent:

‘D’—Draped Fiber, very flexible and lays flat when draped on a spatula.

‘E’—Typical stainless steel laboratory spatula

‘F’—Surface topography variagate, striated and varied.

### Example 4

Structure and function of axial tendons in tunas—The great lateral tendons in tunas and some other scombid fish link mytotomal muscle directly to the caudal fin rays, and thus serve to transfer muscle power to the hydrofoil-like tail during swimming.

### Chemical Characterization

These robust collagenous tendons have structural and mechanical similarity to tendons found in other vertebrates. Biochemical studies indicate that tuna tendon collagen is composed of the two alpha 1(I) chains and one alpha 2(I) chain heterotrimer that is typical of vertebrate type I collagen, while tuna skin collagen has the unusual two alpha 1(I) chains and one alpha 2(I) chain and alpha-3 heterotrimer previously described in other fish skin.

Engineering Characterization (Material Properties)

While changes in covalent crosslinking could be introduced with in vitro incubation over several months (as with mammalian tendons), no differences were detected from fish ranging from 2-75 kg. Application of buckle-type force transducers on caudal tendons in skipjack and yellowfin yielded measurements of in vivo forces during steady and burst swimming. Tendons excised post-mortem were subjected to load cycling to determine the modulus of elasticity and energy dissipation (0.65-1.2 GPa and 7-25% respectively). These material properties compare closely to those of mammalian leg tendons that are known to function as effective biological springs in terrestrial locomotion. However, peak forces doing steady swimming impose strains of much less than 1% of tendon length because the tendons are relatively thick. Even the maximal burst forces recorded produced strains of only 1.5-2%. Consequently, the strain energy stored in the stretched tendon is insignificant compared to the work done by the muscle in producing thrust. Thus, the caudal tendons in tunas do not function as energy saving locomotor springs, even at maximal effort.

### Example 5

RATS: samples cca 15×15 mm subcutaneously on the back. Only bovine tendon collagen sponge and fish collagen it is not clear subcutaneous space, more under external muscles. Twenty rats were studied over a 5-week period, ten rats had bovine tendon collagen sponge implanted subcutaneously and ten rats had fish tendon collagen sponge implanted. Two rats were sacrificed in each group every week and histological examination of the tissue was conducted on the rats sacrificed. The results are as follows:

Bovine collagen:
1 day: Absolutely no reaction in the material.
1 week: Absolutely areactive material under external muscular layer
2 weeks: Absolute areactivity stays. In vicinity minimal fibroproductive reaction, no polynuclears or giant-cells.
3 weeks: Not evaluable.
4 weeks: the original picture from 2nd week remains. Minimal atrophic changes in surrounding muscular tissue. No original sample found.
Fish-collagen:
1 day: Under externally muscular layer. Absolutely no reaction.
1 week: Polynuclears in the external parts of the sponge, internal part without any reaction. Apart from the sponge is accumulation of fibroblasts and fibrin. No giant-cells.
2 weeks: In the centre of a sponge probably necrosis. In the tissue relatively big quantity of nonspecific granulative tissue without polynuclears but with significant development of giant-cells
3 weeks: Similar picture as after two weeks with substantial decrease in the count of giant-cells. Lining by lymphopids.
4 weeks: Original material not found. Fibroproductive reaction of medium intensity, insignificant edema. No giant cells or lymphoids.

It was observed that both bovine collagen and fish collagen were essentially equivalent and each collagen matrix allowed tissue re-growth and complete resorption was noted in 4 to 5 weeks.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A collagen product derived from an animal, the collagen product comprising precipitated collagen from an acidic collagen dispersion, wherein the precipitated collagen is substantially pure collagen.

2. A collagen product according to claim 1, wherein the collagen is from about 98% to about 99% pure collagen.

3. A collagen product according to claim 1, wherein the precipitated collagen comprises two alpha 1(I) chains and one alpha 2(I) chain heterotrimer of collagen or type I collagen and is not derived from skin of an animal.

4. A collagen product according to claim 1, wherein the precipitated collagen comprises in mole percent about: hydroxyproline 7.192, aspartic acid 3.634, threonine 3.303, serine 2.507, glutamic acid 9.353, proline 17.134, glycine 30, alanine 19.598, valine 2.922, methionine 1.711, isoleucine 1.034, leucine 2.356, tyrosine 0.315, phenylalanine 1.291, hydroxylysine 0.437, histidine 0.906, and lysine 4.511.

5. A collagen product according to claim 1, wherein the precipitated collagen comprises a collagen fiber or gelatin.

6. A collagen product according to claim 1, wherein the animal is at least one bird, hoofed mammal, mammal without hoofs; marsupials, amphibian, reptile or marine animal.

7. A collagen product derived from an animal without hoofs, wherein the collagen product comprises two alpha 1(I) chains and one alpha 2(I) chain heterotrimer of collagen or type I collagen and is not derived from skin of the animal.

8. A collagen product according to claim 7, wherein the collagen comprises in mole percent about: hydroxyproline 7.192, aspartic acid 3.634, threonine 3.303, serine 2.507, glutamic acid 9.353, proline 17.134, glycine 30, alanine 19.598, valine 2.922, methionine 1.711, isoleucine 1.034, leucine 2.356, tyrosine 0.315, phenylalanine 1.291, hydroxylysine 0.437, histidine 0.906, and lysine 4.511.

9. A collagen product according to claim 7, wherein the animal is a marine animal, which is a teleost.

10. A collagen product according to claim 9, wherein the teleost is at least one of swordfish, tuna, shark, mahimahi, sailfish, marlin, yellowtail, escolar, lancet fish, mackerel, flounder, carp, salmon, cod, bass, or sturgeon.

11. A collagen product according to claim 9, wherein the collagen is derived from at least one caudal tendon, pectoral tendon, caudal ray tendon, or intercostal tendon of the marine animal.

12. A collagen product according to claim 11, wherein the tendon is at least one caudal tendon, pectoral tendon, caudal ray tendon, or intercostal tendon of a tuna or shark.

13. A collagen product according to claim 1, wherein the collagen is a collagen hydrolysate.

14. A collagen product according to claim 1, which comprises from 0.01% to 100% by weight of collagen.

15. A collagen product according to claim 1, wherein the collagen is deodorized.

16. A collagen product according to claim 1, wherein the collagen has been crosslinked by thermal dehydration, chemical treatment, and/or light.

17. A collagen product according to claim 16, wherein the thermal dehydration is carried under vacuum at a temperature between 60° C. to about 130° C.

18. A collagen product according to claim 1, wherein the collagen product is at least one collagen film, collagen membrane, cosmetic collagen mask, collagen sponge, gelatin, microfibrillar collagen, hemostasis sponge, lyophilized foam, collagen injection, artificial dura or artificial skin.

19. A collagen product according to claim 1, wherein the collagen product is incorporated on or into at least one bone, cartilage, skin, screw, shaft, stent, or tube guide.

20. A collagen product of claim 18, wherein the collagen product is prepared in the form of a collagen film and dried in a stream of air or by lyophilization.

21. A method for obtaining a collagen product from a marine animal comprising: a) isolating two alpha 1(I) chains and one alpha 2(I) chain heterotrimer of collagen or type I collagen from a marine animal, wherein the collagen is not isolated from skin; and b) recovering the two alpha 1(I) chains and one alpha 2(I) chain heterotrimer of collagen or type I collagen to obtain the collagen product.

22. A method for obtaining a collagen product according to claim 21, wherein the collagen comprises in mole percent about: hydroxyproline 7.192, aspartic acid 3.634, threonine 3.303, serine 2.507, glutamic acid 9.353, proline 17.134, glycine 30, alanine 19.598, valine 2.922, methionine 1.711, isoleucine 1.034, leucine 2.356, tyrosine 0.315, phenylalanine 1.291, hydroxylysine 0.437, histidine 0.906, and lysine 4.511.

23. A method for obtaining a collagen product according to claim 21, wherein the collagen is derived from at least one caudal tendon, pectoral tendon, caudal ray tendon, or intercostal tendon of a marine animal.

24. A method for obtaining a collagen product according to claim 23, wherein the tendon is at least one caudal tendon, pectoral tendon, caudal ray tendon, or intercostal, tendon from a tuna or shark.

25. A method for obtaining a collagen product from an animal, comprising: alkalinizing an acidic collagen dispersion containing collagen from the animal; and neutralizing the alkalinized collagen dispersion to precipitate the collagen; and recovering the collagen to obtain the collagen product.

26. A method according to claim 25, wherein the collagen is in the form of a collagen fiber and is from about 98% to about 99% pure collagen.

27. A method according to claim 24, wherein the collagen comprises two alpha 1(I) chains and one alpha 2(I) chain heterotrimer of collagen or type I collagen and is not derived from skin of an animal.

28. A method according to claim 27, wherein the collagen is derived from at least one caudal tendon, pectoral tendon, caudal ray tendon, or intercostal tendon of a marine animal.

29. A method for obtaining collagen fibers from an animal, comprising: a) adding an enzyme to collagen particles obtained from the animal so as to substantially remove non-collagenous materials from the collagen particles, b) inactivating and washing the enzyme from the collagen particles; c) alkalinizing the collagen particles and neutralizing the alkalinized collagen particles with an acid to obtain a collagen dispersion, d) precipitating collagen fibers from the collagen dispersion to obtain the collagen fibers.

30. A method according to claim 29, wherein the collagen fibers are from about 98% to about 99% pure collagen.

31. A method according to claim 29, wherein the collagen fiber are not derived from skin of the animal.

32. A method according to claim 29, wherein the collagen fibers are derived from at least one caudal tendon, pectoral tendon, caudal ray tendon, or intercostal tendon of a marine animal.

33. A method according to claim 29, wherein the collagen fibers are made or incorporated into a collagen product comprising at least one collagen film, collagen membrane, cosmetic collagen mask, gelatin, collagen sponge, microfibrillar collagen, hemostasis sponge, lyophilized foam, collagen injection, artificial dura, or artificial skin.

34. A method according to claim 29, wherein the collagen fibers are incorporated into at least one bone, cartilage, skin, screw, shaft, stent, or tube guide.

35. A method according to claim 29, wherein step c) further comprises separating the collagen particles from non-collagenous materials.
